(19) United States
(12) Patent Application Publication
(10) Pub. No.: US 2018/0314077 A1
(43) Pub. Date: Nov. 1, 2018

(54) DRUG SUSTAINED-RELEASING MEDICAL CONTACT LENS

(71) Applicant: NOF CORPORATION, Shibuya-ku, Tokyo (JP)

(72) Inventors: Shunsuke Sakurai, Kawasaki-shi, Kanagawa (JP); Kei Sato, Kawasaki-shi, Kanagawa (JP); Masatomo Takahashi, Kawasaki-shi, Kanagawa (JP); Mitsuyasu Nakashima, Kawasaki-shi, Kanagawa (JP); Yosuke Matsuoka, Kawasaki-shi, Kanagawa (JP); Yoshihisa Shimamura, Kawasaki-shi, Kanagawa (JP); Koji Miyamoto, Kawasaki-shi, Kanagawa (JP); Nobuyuki Yamamoto, Kawasaki-shi, Kanagawa (JP)

(73) Assignee: NOF CORPORATION, Shibuya-ku, Tokyo (JP)

(21) Appl. No.: 15/770,292

(22) PCT Filed: Oct. 28, 2016
(86) PCT No.: PCT/JP2016/082093

§ 371 Date: Apr. 23, 2018

(30) Foreign Application Priority Data
Oct. 30, 2015 (JP) 2015-214842

(51) Int. Cl. G02C 7/04; A61L 27/44; A61L 27/54; C08F 30/08

(52) U.S. Cl. CPC G02C 7/049; C08F 30/08; A61L 27/54; A61L 27/44

(57) ABSTRACT

Provided is a drug sustained-releasing medical contact lens in which a component that is water-insoluble and has pharmacological activity is retained to impart drug sustained-releasability, and which is excellent in oxygen permeability. It has been found that a drug sustained-releasing medical contact lens containing a specific amount of each of a silicone-based polymer having a specific structure and a drug having a specific solubility can achieve the above-mentioned contact lens.

### TECHNICAL FIELD

The present invention relates to a drug sustained-releasing medical contact lens, and more specifically, to a drug sustained-releasing medical contact lens optimum for a hard contact lens or a soft contact lens out of contact lenses.

The present application claims priority from Japanese Patent Application No. 2015-214842, which is incorporated herein by reference.

### BACKGROUND ART

The performance of treatment with an eye drop that is easy to handle first has been generally considered to be a first choice for disease treatment in the field of ophthalmology. Although the eye drop is easy to handle, a drug dropped to an eye follows the following route. The drug is instantaneously diluted by a lacrimal fluid and a nictitation, and is discharged from a lacrimal point. It has heretofore been reported that about 99% of drugs administered in drops to eyes do not reach the insides of eye sockets (Non Patent Literature 1), and it has been known that it is extremely difficult to retain the drug dropped to the eye in a conjunctival sac over a long time period. Accordingly, in order that the migrating property of the drug to the inside of an eye socket may be improved, for example, the following methods are conceivable. The number of times of dropping of the drug to the eye per day is increased or the amount of the drug to be dropped to the eye per dropping is increased. However, both the methods have not been considered to be better treatment methods because the risks of side effects increase.

In view of the foregoing, many investigations have heretofore been made on a technology involving, for the purpose of retaining a drug in the conjunctival sac over a long time period, introducing and retaining the drug in a contact lens in advance, and sustained-releasing the retained drug (Patent Literature 1, Patent Literature 2, and Non Patent Literature 2). As a result of those investigations, there has been reported a possibility that the drug is surely retained in the contact lens over a long time period, and hence sustained-releasability is imparted to the contact lens to enable its effective utilization in treatment. However, the investigated drug is limited to a water-soluble drug (Patent Literature 1, Patent Literature 2, and Non Patent Literature 2). This is probably because of the following reason. In ordinary cases, the contact lens is often stored in water, such as a preserving liquid, and hence mixing with a water-insoluble compound may cause cloudiness, precipitation, or the like to make it impossible to use the contact lens. Accordingly, the water-soluble drug is frequently used.

However, about one half of components each having pharmacological activity to be used in the eye drop are water-soluble, and about the other half thereof are water-insoluble. For example, latanoprost serving as a water-insoluble component is a typical drug for glaucoma treatment, and has been widely used in Japan. When, as described above, a water-insoluble drug is retained in a contact lens and hence the contact lens can have drug sustained-releasing performance, it is assumed that the range of choices for treatment extends and hence a better treatment method can be provided.

Meanwhile, when a contact lens having drug sustained-releasability is used in treatment, the contact lens is worn over a long time period. Accordingly, in today's circumstances, the contact lens having the drug sustained-releasability is required to have better oxygen permeation performance as well. It has been generally known that the oxygen permeability of a contact lens is improved by blending the contact lens with a silicone monomer. A silicone monomer excellent in oxygen permeability and hydrophilicity on the surface of a contact lens has heretofore already been developed (Patent Literature 3), but whether or not the use of the silicone monomer imparts drug sustained-releasability to the contact lens has not been known at all.

As described above, the development of a contact lens composition that can retain and sustained-release a water-insoluble effective component over a long time for a long period, and that is excellent in oxygen permeability has been craved.

### CITATION LIST

### Patent Literature

[PTL 1] WO 2012/127927 A1
[PTL 2] WO 2003/090805 A1
[PTL 3] WO 2010/082659 A1 Non Patent Literature
[NPL 1] M. Patrick and A. K. Mitra, Overview of Ocular Drug Delivery and Iatrogenic Ocular Cytopathologies, Ophthalmic Drag Delivery Systems, 1-27, Marcel Dekker, New York, 1993.
[NPL 2] H. Hiratani and C. A. Lorenzo, Timolol uptake and release by imprinted soft contact lense made of N,N-diethylacrylamide and methacrylic acid, J. Control Release, 83, 223-230, 2002.

### SUMMARY OF INVENTION

### Technical Problem

An object of the present invention is to provide a drug sustained-releasing medical contact lens in which a component that is water-insoluble and has pharmacological activity is retained to impart drug sustained-releasability, and which is excellent in oxygen permeability.

### Solution to Problem

The inventors of the present invention have made extensive investigations for achieving the object, and as a result, have found that a drug sustained-releasing medical contact lens containing a specific amount of each of a silicone-based polymer having a specific structure and a drug having a solubility in a specific range can achieve the object. Thus, the inventors have completed the present invention.

That is, the present invention provides the following items [1] to [8].

[1] A drug sustained-releasing medical contact lens, including: a polymer (P) having a constituent unit represented by the formula (1); and a drug (Q) having a solubility in water of from 0.00001% to 3.3%, in which a content of the polymer (P) is from 90 mass % to 99.99999 mass %, and a content of the drug (Q) is from 0.00001 mass % to 10 mass %:

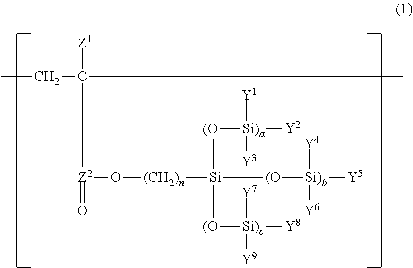

where Y¹ to Y⁹ each independently represent an alkyl group having 1 to 4 carbon atoms, n represents an integer of from 0 to 3, a, b, and c each independently represent an integer of 0 or 1, Z¹ represents a hydrogen atom, a methyl group, or CH₂—C(═O)—X, X represents a monovalent organic group having 2 to 6 carbon atoms that contain one or more hydroxyl groups, and that contain one oxygen atom, and that may contain nitrogen atom in a main chain thereof, and Z² represents a carbon atom or C(═O)—O—(CH₂)₂—O—C(═O)—(CH₂)₂—C.

[2] A drug sustained-releasing medical contact lens according to the above-mentioned item [1], in which the constituent unit represented by the formula (1) is represented by the formula (A) or the formula (B):

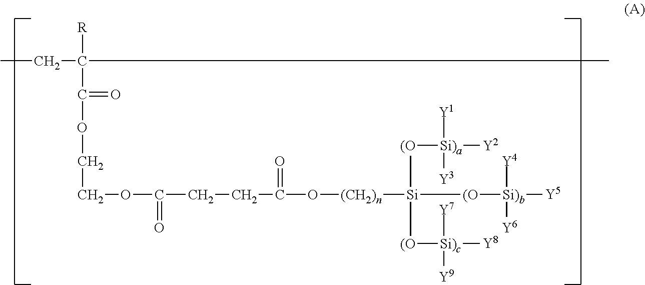

where Y¹ to Y⁹ each independently represent an alkyl group having 1 to 4 carbon atoms, n represents an integer of from 0 to 3, a, b, and c each independently represent an integer of 0 or 1, and R represents a hydrogen atom or a methyl group;

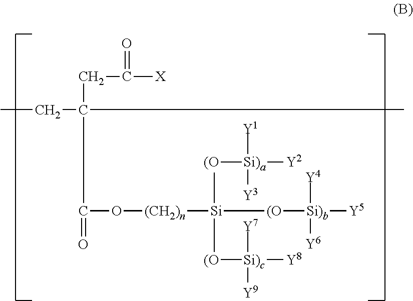

where Y¹ to Y⁹ each independently represent an alkyl group having 1 to 4 carbon atoms, n represents an integer of from 0 to 3, a, b, and c each independently represent an integer of 0 or 1, and X represents a monovalent organic group having 2 to 6 carbon atoms that contain one or more hydroxyl groups, and that contain one oxygen atom, and that may contain nitrogen atom in a main chain thereof.

[3] A drug sustained-releasing medical contact lens according to the above-mentioned item [1] or [2], in which X represents any one of the following groups (a) to (f).

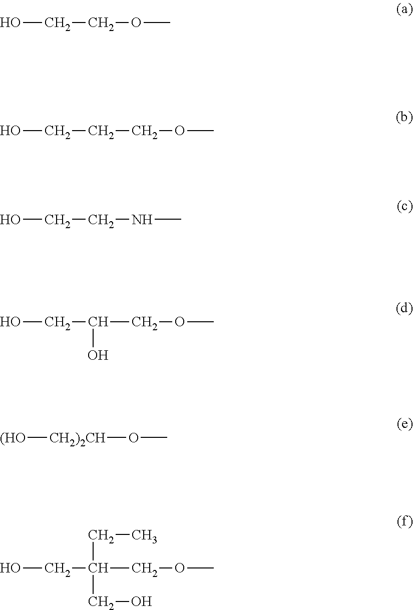

[4] A drug sustained-releasing medical contact lens according to any one of the above-mentioned items [1] to [3], in which the drug (Q) includes one or more kinds of drug (Q) selected from the group consisting of a drug for glaucoma treatment, an antiallergic drug, an antiinflammatory drug, an adrenocortical steroid drug, an antibacterial drug, an anti-cataract drug, a drug for corneal disorder treatment, an antiviral drug, a mydriatic, a cycloplegic, and a vitamin agent.

[5] A drug sustained-releasing medical contact lens according to the above-mentioned item [1], in which the polymer (P) includes a polymer having a constituent unit based on 3-[tris(trimethylsiloxy)silyl]propyl methacryloyloxyethylsuccinate, and the drug (Q) includes latanoprost.

[6] A drug sustained-releasing medical contact lens according to the above-mentioned item [1], in which the polymer (P) includes a polymer having a constituent unit based on [tris(trimethylsiloxy)silyl]propyl=3-(2-hydroxyethoxy)carbonyl-3-butenate, and the drug (Q) includes latanoprost.

[7] A drug sustained-releasing method, including using a drug sustained-releasing medical contact lens in which a content of a polymer (P) having a constituent unit represented by the formula (1) is from 90 mass % to 99.99999 mass %, and a content of a drug (Q) having a solubility in water of from 0.00001% to 3.3% is from 0.00001 mass % to 10 mass % in a mammal including a human:

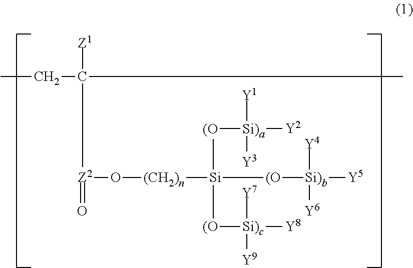

where Y¹ to Y⁹ each independently represent an alkyl group having 1 to 4 carbon atoms, n represents an integer of from 0 to 3, a, b, and c each independently represent an integer of 0 or 1, Z¹ represents a hydrogen atom, a methyl group, or CH₂—C(═O)—X, X represents a monovalent organic group having 2 to 6 carbon atoms that contain one or more hydroxyl groups, and that contain one oxygen atom, and that may contain nitrogen atom in a main chain thereof, and Z² represents a carbon atom or C(═O)—O—(CH₂)₂—O—C(═O)—(CH₂)₂—C.

[8] A method of using a polymer (P) having a constituent unit represented by the formula (1) and a drug (Q) having a solubility in water of from 0.00001% to 3.3% for producing a drug sustained-releasing medical contact lens in which a content of the polymer (P) is from 90 mass % to 99.99999 mass %, and a content of the drug (Q) is from 0.00001 mass % to 10 mass %:

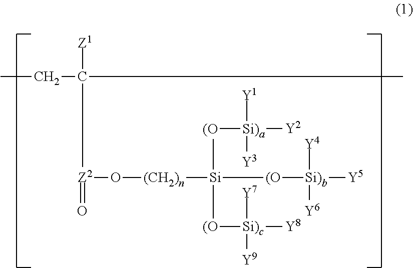

where Y¹ to Y⁹ each independently represent an alkyl group having 1 to 4 carbon atoms, n represents an integer of from 0 to 3, a, b, and c each independently represent an integer of 0 or 1, Z¹ represents a hydrogen atom, a methyl group, or CH₂—C(═O)—X, X represents a monovalent organic group having 2 to 6 carbon atoms that contain one or more hydroxyl groups, and that contain one oxygen atom, and that may contain nitrogen atom in a main chain thereof, and Z² represents a carbon atom or C(═O)—O—(CH₂)₂—O—C(═O)—(CH₂)₂—C.

### Advantageous Effects of Invention

The drug sustained-releasing medical contact lens of the present invention is effective in improving the intraocular migrating property of the drug (Q) because the drug (Q) can be retained in the contact lens, and the drug (Q) can be sustained-released at the time of the wearing of the contact lens. In addition, the drug sustained-releasing medical contact lens of the present invention shows excellent oxygen permeability because the contact lens has the constituent unit containing a silicone moiety at a specific ratio.

### BRIEF DESCRIPTION OF DRAWINGS

FIG. 1 is a graph for showing the results of the contact lens drug sustained-releasability tests of Example 5 and Example 6. The axis of ordinate indicates a drug sustained-release ratio (%), and the axis of abscissa indicates an immersion time (h) in ISO physiological saline (phosphate buffer specified in ISO 18369-3). The results of Example 5 are represented by ⋄ and the results of Example 6 are represented by □.

FIG. 2 is a graph for showing the results of the contact lens drug sustained-releasability tests of Example 7 to Example 10. The axis of ordinate indicates a drug sustained-release ratio (%), and the axis of abscissa indicates an immersion time (h) in ISO physiological saline. The results of Example 7 are represented by ⋄, the results of Example 8 are represented by □, the results of Example 9 are represented by Δ, and the results of Example 10 are represented by x.

### DESCRIPTION OF EMBODIMENTS

The present invention is described in more detail below.

A drug sustained-releasing medical contact lens of the present invention contains a polymer (P) and a drug (Q) described below.

<Polymer (P)>

The polymer (P) to be used in the present invention has a constituent unit represented by the following formula (1), and the constituent unit more specifically has a constituent unit represented by the formula (A) or the formula (B).

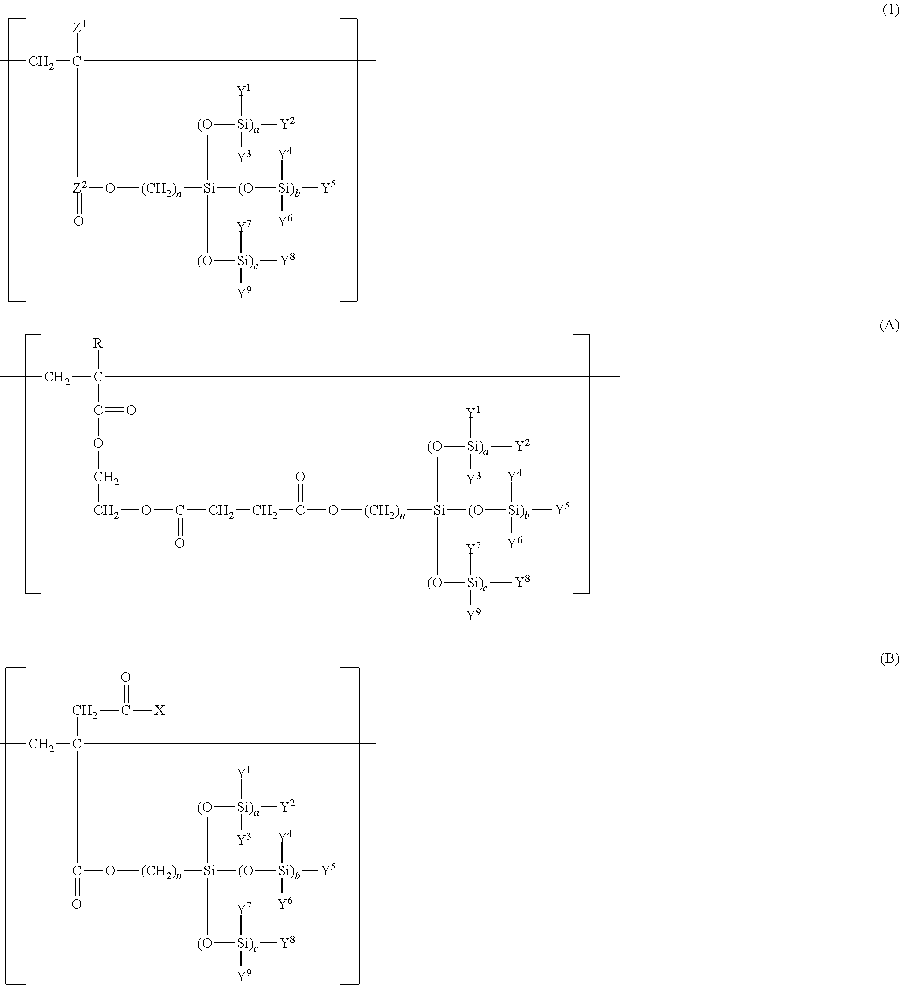

The monomer of the constituent unit represented by the formula (A) is represented by the following formula (C). In addition, the monomer of the constituent unit represented by the formula (B) is represented by the following formula (D1) or the following formula (D2)

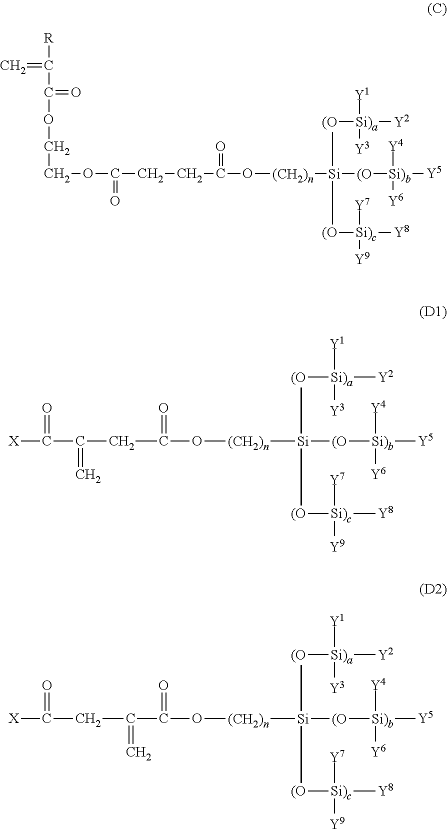

In each of the formula (C), the formula (D1), and the formula (D2), Y¹ to Y⁹ each independently represent an alkyl group having 1 to 4 carbon atoms. Examples of the alkyl group having 1 to 4 carbon atoms include a methyl group, an ethyl group, a n-propyl group, an isopropyl group, a n-butyl group, and a t-butyl group. Of those, a methyl group is preferred from the viewpoint of improving the oxygen permeability of the drug sustained-releasing medical contact lens.

R represents a hydrogen atom or a methyl group. Of those, a methyl group is preferred.

n represents an integer of from 0 to 3. n preferably represents 3 from the viewpoint of improving the oxygen permeability.

a, b, and c each independently represent an integer of 0 or 1. X represents a monovalent organic group having 2 to 6 carbon atoms that contain one or more hydroxyl groups, and that contain one oxygen atom, and that may contain nitrogen atom in a main chain thereof. Examples of X serving as a monovalent organic group include groups represented by the formula (a) to the formula (f).

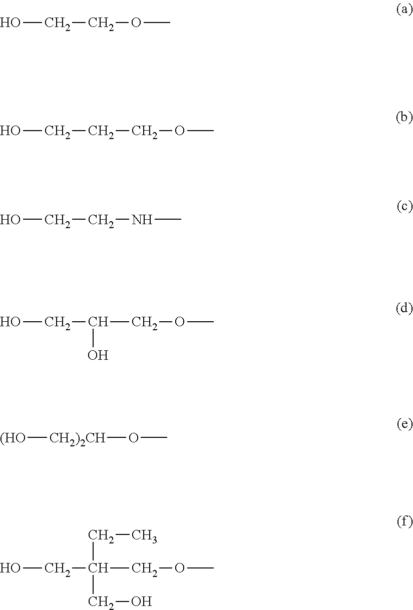

From the viewpoint of improving the oxygen permeability, a compound represented by the formula (E) in which in the formula (C), a, b, and c each represent 1, n represents 3, and R represents a methyl group (compound name: 3-[tris(trimethylsiloxy)silyl]propyl methacryloyloxyethylsuccinate), by the formula (F1) in which in the formula (D2), a, b, and c each represent 1, n represents 3, and X represents the formula (a) (compound name: [tris(trimethylsiloxy)silyl]propyl=3-(2-hydroxyethoxy)carbonyl-3-butenate), or by the formula (F2) in which in the formula (D2), a, b, and c each represent 1, n represents 3, and X represents the formula (c) (compound name: 2-[N-(2-hydroxyethyl)carbamoylmethyl]-3-[tris(trimethylsiloxy) silyl]propyl acrylate) is particularly preferred. In each of the formula (E), the formula (F1), and the formula (F2), Y¹ to Y⁹ each represent a methyl group.

In other words, in terms of a polymer, the constituent unit represented by the formula (1) in the polymer (P) is particularly preferably a constituent unit based on 3-[tris(trimethylsiloxy)silyl]propyl methacryloyloxyethylsuccinate, a constituent unit based on [tris(trimethylsiloxy)silyl]propyl=3-(2-hydroxyethoxy)carbonyl-3-butenate, or a constituent unit based on 2-[N-(2-hydroxyethyl) carbamoylmethyl]-3-[tris(trimethylsiloxy) silyl]propyl acrylate.

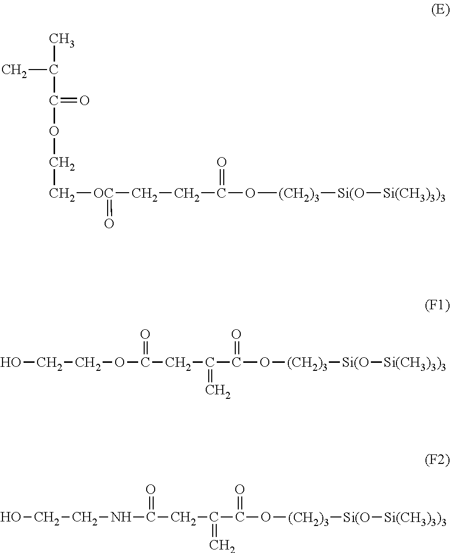

The constituent unit (formula (1)) containing a silicone moiety to be used in the drug sustained-releasing medical contact lens of the present invention is specifically represented by the formula (A) or the formula (B), the formula (A) is obtained by polymerizing the monomer represented by the formula (C), and the monomer represented by the formula (C) may be synthesized in accordance with a method described in WO 2010/082659 A1. The formula (B) is obtained by polymerizing the monomer represented by the formula (D1) or the formula (D2), and the monomer represented by the formula (D1) or the formula (D2) may be synthesized in accordance with a method described in WO 2010/104000 A1.

The constituent unit (formula (1)) containing the silicone moiety to be used in the drug sustained-releasing medical contact lens of the present invention can be used in a medical lens, such as a hard contact lens, a soft contact lens, or an intraocular lens. The constituent unit is preferably used in the hard contact lens or the soft contact lens out of such lenses, and is particularly preferably used in the soft contact lens.

The polymer (P) to be incorporated into the drug sustained-releasing medical contact lens of the present invention may be formed only of the constituent unit (formula (1)) containing the silicone moiety, but typically contains a constituent component except the formula (1). When the constituent component except the formula (1) is used in the drug sustained-releasing medical contact lens, a copolymer obtained by polymerizing the monomer represented by the formula (C), the formula (D1), or the formula (D2) (the monomer of the constituent unit represented by the formula (1)), and a monomer except the foregoing (hereinafter sometimes referred to as “other monomer”) may be used. A ratio between the monomer represented by the formula (C), the formula (D1), or the formula (D2), and the other monomer falls within the range of from 1:9 to 8:2 in terms of a mass ratio.

In more detail, the polymer (P) to be incorporated into the drug sustained-releasing medical contact lens of the present invention is obtained by polymerizing a mixture containing 10 mass % to 80 mass %, preferably 20 mass % to 70 mass %, more preferably 30 mass % to 70 mass % of the monomer of the constituent unit represented by the formula (1), and 90 mass % to 20 mass %, preferably 80 mass % to 30 mass %, more preferably 70 mass % to 30 mass % of the other monomer.

The content of the constituent unit represented by the formula (1) in the copolymer to be used in the present invention is preferably from 1 mol % to 50 mol %, more preferably from 5 mol % to 40 mol %.

A monomer appropriately selected from monomers generally used as monomers to be used in contact lenses may be used as the other monomer to be used in the drug sustained-releasing medical contact lens of the present invention.

Examples of the other monomer to be used in the drug sustained-releasing medical contact lens of the present invention include water-soluble monomers, such as (meth)acrylic acid, itaconic acid, crotonic acid, cinnamic acid, vinylbenzoic acid, 2-(meth)acryloyloxyethyl phosphorylcholine, a polyalkylene glycol mono(meth)acrylate, a polyalkylene glycol monoalkyl ether (meth)acrylate, 2-hydroxyethyl (meth)acrylate, 2,3-dihydroxypropyl (meth)acrylate, glycerol (meth)acrylate, N-vinylformamide, N-vinylacetamide, N-methyl-N-vinylacetamide, and N-vinylpyrrolidone, with intent to enhance the surface hydrophilicity of the contact lens. Of those, 2-hydroxyethyl (meth)acrylate is particularly preferred from the viewpoint of enhancing the surface hydrophilicity of the contact lens.

Examples of the other monomer to be used in the drug sustained-releasing medical contact lens of the present invention include a polyalkylene glycol bis(meth)acrylate, trimethylolpropane tris(meth)acrylate, pentaerythritol tetrakis (meth)acrylate, N,N-dimethylacrylamide, N,N-diethylacrylamide, N,N-di-n-propylacrylamide, N,N-diisopropylacrylamide, N,N-di-n-butylacrylamide, N-acryloylmorpholine, N-acryloylpiperidine, N-vinylcaprolactam, N-vinyloxazolidone, 1-vinylimidazole, N-vinylcarbazole, vinylpyridine, and vinylpyrazine with intent to control the flexibility of the contact lens.

Examples of the other monomer to be used in the drug sustained-releasing medical contact lens of the present invention include: alkyl (meth)acrylates, such as methyl (meth)acrylate and ethyl (meth)acrylate; polyfunctional (meth)acrylates, such as a siloxane macromonomer having carbon-carbon unsaturated bonds at both terminals thereof and ethylene glycol dimethacrylate; trifluoroethyl (meth)acrylates; aromatic vinyl monomers, such as styrene, α-methylstyrene, and vinylpyridine; and vinyl esters, such as vinyl acetate, with intent to improve the shape-maintaining property of the contact lens.

The drug sustained-releasing medical contact lens of the present invention may be blended with any of the following monomers in addition to the monomer intended to enhance the surface hydrophilicity of the drug sustained-releasing medical contact lens of the present invention, the monomer intended to control the flexibility thereof, and the monomer intended to improve the shape-maintaining property thereof. Examples thereof include 3-[tris(trimethylsiloxy)silyl]propyl (meth)acrylate, 3-[bis(trimethylsiloxy)methylsilyl]propyl (meth)acrylate, 3-[(trimethylsiloxy)dimethylsilyl]propyl (meth)acrylate, 3-[tris(trimethylsiloxy)silyl]propyl (meth)acrylamide, 3-[bis(trimethylsiloxy)methylsilyl]propyl (meth)acrylamide, 3-[(trimethylsiloxy)dimethylsilyl]propyl (meth)acrylamide, [tris(trimethylsiloxy)silyl]methyl (meth)acrylate, [bis(trimethylsiloxy)methylsilyl]methyl (meth)acrylate, [(trimethylsiloxy)dimethylsilyl]methyl (meth)acrylate, [tris(trimethylsiloxy)silyl]methyl (meth)acrylamide, [bis(trimethylsiloxy)methylsilyl]methyl (meth)acrylamide, [(trimethylsiloxy)dimethylsilyl]methyl (meth)acrylamide, [tris(trimethylsiloxy)silyl]styrene, [bis(trimethylsiloxy)methylsilyl]styrene, [(trimethylsiloxy)dimethylsilyl]styrene, N-[3-[tris(trimethylsiloxy)silyl]propyl] vinyl carbamate, N-[3-[bis(trimethylsiloxy)methylsilyl]propyl] vinyl carbamate, and N-[3-[(trimethylsiloxy)dimethylsilyl]propyl] vinyl carbamate.

The drug sustained-releasing medical contact lens of the present invention may be produced by: mixing the monomer (C), monomer (D1), or monomer (D2) of the constituent unit represented by the formula (1), and the other monomer; and appropriately adding a thermal polymerization initiator typified by a peroxide or an azo compound, or a photopolymerization initiator to the mixture. When thermal polymerization is performed, a thermal polymerization initiator having a decomposition characteristic optimum for a desired reaction temperature may be selected and used. That is, a peroxide or an azo compound having a 10-hour half-life temperature of from 40° C. to 120° C. is preferably used. Examples of the azo compound may include 2,2-azobis(2-aminopropyl) dihydrochloride, 2,2-azobis(2-(5-methyl-2-imidazolin-2-yl)propane) dihydrochloride, 4,4-azobis(4-cyanovaleric acid), 2,2-azobisisobutylamide dihydrate, 2,2-azobis(2,4-dimethylvaleronitrile), 2,2-azobisisobutyronitrile (azobisisobutyronitrile, AIBN), dimethyl-2,2′-azobisisobutyrate, 1-((1-cyano-1-methylethyl)azo)formamide, 2,2′-azobis(2-methyl-N-phenylpropionamidine) dihydrochloride, 2,2′-azobis(2-methyl-N-(2-hydroxyethyl)-propionamide), 2,2′-azobis(2-methylpropionamide) dihydrate, 4,4′-azobis(4-cyanopentanoic acid), and 2,2′-azobis(2-(hydroxymethyl)propionitrile). Examples of the photopolymerization initiator may include a carbonyl compound, a sulfur compound, a halogen compound, and a metal salt. Those polymerization initiators may be used alone or in combination thereof. The polymerization initiator is preferably used at a ratio of from 0.2 part by mass to 2 parts by mass with respect to 100 parts by mass of the components to be polymerized.

The content of the polymer (P) to be used in the drug sustained-releasing medical contact lens of the present invention is from 90 mass % to 99.99999 mass %, and is preferably from 93 mass % to 99.99998 mass %, more preferably from 95 mass % to 99.99997 mass % from the viewpoints of the drug sustained-releasability of the contact lens and an improvement in oxygen permeability thereof.

<Drug (Q)>

The drug (Q) to be used in the drug sustained-releasing medical contact lens of the present invention is a component having pharmacological activity to be used in an ophthalmic drug, and the solubility of such component having pharmacological activity in water is from 0.00001% to 3.3%. The solubility of the component having pharmacological activity in water is preferably from 0.00001% to 2.9%, more preferably from 0.00001% to 2.8% from the viewpoint of compatibility between the constituent unit (1) blended with the silicone moiety to be used in the drug sustained-releasing medical contact lens of the present invention and the component having pharmacological activity.

The solubility of the drug (Q) to be used in the drug sustained-releasing medical contact lens of the present invention in water represents the mass (g) of water at 25° C. needed for dissolving the drug (Q) when the water is added to 0.01 g of the drug (Q). The solubility of the drug (Q) to be used in the drug sustained-releasing medical contact lens of the present invention in water may be calculated by using the following equation.

(Solubility in water) (%)=(0.01 (g) of drug (Q))/(mass (g) of water needed for dissolving the drug,25° C.)×100

The drug (Q) to be used in the drug sustained-releasing medical contact lens of the present invention is preferably one or more kinds of drug (Q) selected from the group consisting of an antiallergic drug, a drug for glaucoma treatment, an antiinflammatory drug, an adrenocortical steroid drug, an antibacterial drug, an anti-cataract drug, a drug for corneal disorder treatment, an antiviral drug, a mydriatic, a cycloplegic, and a vitamin agent. More specific examples of the drug (Q) include the following drugs. In addition, after the name of each drug (Q), the solubility of the drug (Q) in water is described in parentheses.

Examples of the antiallergic drug include amlexanox (0.01%), tranilast (0.01%), levocabastine hydrochloride (0.01%), acitazanolast hydrate (0.55%), ketotifen fumarate (0.55%), and olopatadine hydrochloride (2.15%).

Examples of the drug for glaucoma treatment include isopropyl unoprostone (0.01%), tafluprost (0.01%), travoprost (0.01%), latanoprost (0.01%), brinzolamide (0.055%), bimatoprost (0.55%), and bunazosin hydrochloride (0.55%).

Examples of the antiinflammatory drug include indomethacin (0.00009%), nepafenac (0.01%), pranoprofen (0.01%), diclofenac sodium (0.24%), and sodium azulene sulfonate hydrate (2.15%).

Examples of the adrenocortical steroid drug include dexamethasone (0.01%), hydrocortisone acetate (0.0014%), fluorometholone (0.003%), prednisolone acetate (0.0017%), methylprednisolone (0.01%), and dexamethasone sodium sulfobenzoate (0.55%).

Examples of the antibacterial drug include tosufloxacin tosilate hydrate (0.01%), norfloxacin (0.01%), cefmenoxime (0.055%), lomefloxacin hydrochloride (0.55%), ofloxacin (2.8%), gatifloxacin (0.55%), chloramphenicol (0.25%), and levofloxacin hydrate (2.15%).

An example of the anti-cataract drug is pirenoxine (0.01%).

Examples of the drug for corneal disorder treatment include rebamipide (0.01%) and sodium hyaluronate (2.15%).

An example of the antiviral drug is acyclovir (0.16%).

An example of the mydriatic and/or the cycloplegic is tropicamide (0.55%).

An example of the vitamin agent is cyanocobalamin (2.15%).

Of the drugs (Q) to be used in the drug sustained-releasing medical contact lens of the present invention, the drug for glaucoma treatment is more preferred from the viewpoint of the drug sustained-releasability of the contact lens. Of such drugs for glaucoma treatment, latanoprost is particularly preferred.

The blending amount of the drug (Q) to be used in the drug sustained-releasing medical contact lens of the present invention is from 0.00001 mass % to 10 mass %, and is preferably from 0.00002 mass % to 7 mass %, more preferably from 0.00003 mass % to 5 mass % from the viewpoints of the drug sustained-releasability of the contact lens and an improvement in oxygen permeability thereof.

<Drug Sustained-Releasing Medical Contact Lens>

The drug sustained-releasing medical contact lens of the present invention may be produced by combining steps known to a person skilled in the art. For example, the contact lens may be produced by the following steps, though a production method therefor is not limited.

For example, the drug sustained-releasing medical contact lens of the present invention may be obtained by: mixing the monomer represented by the formula (C), the formula (D1), or the formula (D2), the desired other monomer, and the drug (Q); adding a polymerization initiator to the mixture; stirring and dissolving the contents to provide a monomer mixed liquid; loading the resultant monomer mixed liquid into a desired molding die; performing the copolymerization reaction of the monomer mixed liquid to provide a copolymer; cooling the copolymer; releasing the cooled copolymer from the molding die; cutting and polishing the copolymer as required; and then hydrating and swelling the molded copolymer.

In addition, for example, the drug sustained-releasing medical contact lens of the present invention may be obtained by: mixing the monomer represented by the formula (C), the formula (D1), or the formula (D2), and the desired other monomer; adding a polymerization initiator to the mixture; stirring and dissolving the contents to provide a monomer mixed liquid; loading the resultant monomer mixed liquid into a desired molding die; performing the copolymerization reaction of the monomer mixed liquid to provide a copolymer; cooling the copolymer; releasing the cooled copolymer from the molding die; cutting and polishing the copolymer as required to produce a contact lens; and impregnating the contact lens produced by the polymerization reaction with a liquid having dissolved therein the drug (Q) to retain (infiltrate) the drug (Q) in the contact lens. At this time, water, physiological saline, an organic solvent, or the like may be used as the liquid for dissolving the drug (Q), and the liquid may be blended with a surfactant, an inorganic salt, a salt of an organic acid, an acid, a base, an antioxidant, a stabilizer, or an antiseptic as required. Examples of the surfactant include, but not particularly limited to, benzalkonium chloride and a polysorbate, and examples of the inorganic salt include, but not particularly limited to, sodium chloride, disodium hydrogen phosphate, and sodium dihydrogen phosphate.

Further, for example, the drug sustained-releasing medical contact lens of the present invention may be obtained by: mixing the monomer represented by the formula (C), the formula (D1), or the formula (D2), and the desired other monomer; adding a polymerization initiator to the mixture; stirring and dissolving the contents to provide a monomer mixed liquid; loading the resultant monomer mixed liquid into a desired molding die; performing the copolymerization reaction of the monomer mixed liquid to provide a copolymer; cooling the copolymer; releasing the cooled copolymer from the molding die; cutting and polishing the copolymer as required to produce a contact lens; preparing a liquid having dissolved therein the drug (Q) separately from the contact lens; and applying the liquid to the surface of the contact lens, or coating the surface with the liquid, to retain the drug (Q) in the contact lens. At this time, water, physiological saline, an organic solvent, or the like may be used as the liquid for dissolving the drug (Q), and the liquid may be blended with a surfactant, an inorganic salt, a salt of an organic acid, an acid, a base, an antioxidant, a stabilizer, or an antiseptic as required. Examples of the surfactant include, but not particularly limited to, benzalkonium chloride and a polysorbate, and examples of the inorganic salt include, but not particularly limited to, sodium chloride, disodium hydrogen phosphate, and sodium dihydrogen phosphate.

As another method, the monomer represented by the formula (C), the formula (D1), or the formula (D2), the desired other monomer, and the drug (Q) are mixed to prepare a mixed liquid. The mixed liquid is injected into a molding die, and the clamping of the molding die is performed. Next, the mixed liquid is polymerized by irradiating the molding die with UV light, and then the resultant polymer is removed from the molding die. Further, an unreacted monomer and the drug (Q) present are removed by washing the polymer with a large excess amount of saline for 3 days or more. In order to incorporate the drug (Q) into a contact lens thus obtained again, the contact lens is immersed in an aqueous solution having dissolved or suspended therein the drug (Q) for from 1 hour to several days. Thus, a contact lens having a large drug uptake and capable of sustained-releasing the drug can be obtained (see Patent Literature 2).

Preferred combinations of the polymer (P) and drug (Q) of the contact lens of the present invention are as described below, but are not particularly limited to the following: a combination of (P) a polymer having a constituent unit based on 3-[tris(trimethylsiloxy)silyl]propyl methacryloyloxyethylsuccinate and (Q) latanoprost;

a combination of (P) a polymer having a constituent unit based on [tris(trimethylsiloxy)silyl]propyl=3-(2-hydroxyethoxy)carbonyl-3-butenate and (Q) latanoprost; and

a combination of (P) a polymer having a constituent unit based on 2-[N-(2-hydroxyethyl)carbamoylmethyl]-3-[tris(trimethylsiloxy) silyl]propyl acrylate and (Q) latanoprost.

The present invention is also directed to a drug sustained-releasing method, including using a drug sustained-releasing medical contact lens in which the content of the polymer (P) having the constituent unit represented by the formula (1) is from 90 mass % to 99.99999 mass %, and the content of the drug (Q) having a solubility in water of from 0.00001% to 3.3% is from 0.00001 mass % to 10 mass % in a mammal including a human.

Although the drug sustained-releasing method of the present invention is not particularly limited, for example, the drug sustained-releasing medical contact lens of the present invention can be worn on an eyeball for 60 minutes or more per day.

The present invention is also directed to a method of using the polymer (P) having a constituent unit represented by the formula (1) and the drug (Q) having a solubility in water of from 0.00001% to 3.3% for producing a drug sustained-releasing medical contact lens in which the content of the polymer (P) is from 90 mass % to 99.99999 mass %, and the content of the drug (Q) is from 0.00001 mass % to 10 mass %:

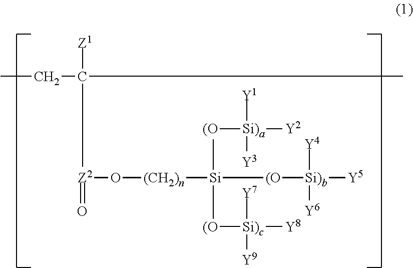

where Y¹ to Y⁹ each independently represent an alkyl group having 1 to 4 carbon atoms, n represents an integer of from 0 to 3, a, b, and c each independently represent an integer of 0 or 1, Z¹ represents a hydrogen atom, a methyl group, or CH₂—C(═O)—X, X represents a monovalent organic group having 2 to 6 carbon atoms that contain one or more hydroxyl groups, and that contain one oxygen atom, and that may contain nitrogen atom in a main chain thereof, and Z² represents a carbon atom or C(═O)—O—(CH₂)₂—O—C(═O)—(CH₂)₂—C.

### EXAMPLES

The present invention and its effects are specifically described by way of the following Examples and Comparative Examples. Silicone monomers used in Examples (3-[tris(trimethylsiloxy)silyl]propyl methacryloyloxyethylsuccinate and [tris(trimethylsiloxy)silyl]propyl=3-(2-hydroxyethoxy)carbonyl-3-butenate) were each obtained by performing synthesis in accordance with a method described in WO 2010/082659 A1 or WO 2010/104000 A1.

### Example 1

40 Parts by mass of 3-[tris(trimethylsiloxy)silyl]propyl methacryloyloxyethylsuccinate {compound represented by the formula (E)}, 60 parts by mass of 2-hydroxyethylmethacrylate (HEMA, other monomer), and 0.005 part by mass of latanoprost {drug (Q)} were mixed to prepare a drug sustained-releasing medical contact lens solution of the present invention. The transparency of the solution was visually observed. The transparency was such that the components were uniformly dissolved without any impurities and the like.

### Example 2

A drug sustained-releasing medical contact lens solution of the present invention was prepared in accordance with the same procedure as that of Example 1 except that the amounts of the monomers to be used were changed. The result of its evaluation is shown in Table 1 together with its composition.

### Comparative Example 1 and Comparative Example 2

Contact lens solutions different from Examples 1 and 2 (drug sustained-releasing medical contact lens solutions) were each prepared in accordance with the same procedure as that of Example 1 or Example 2 except that 3-tris(trimethylsilyl)propyl methacrylate (abbreviated as “TRIS”) that did not correspond to any one of the formula (C), the formula (D1), and the formula (D2) was used instead of the monomer represented by the formula (E). The results of their evaluations are shown in Table 1 together with their compositions.

As a result, in each of Example 1 and Example 2, a transparent solution was able to be prepared at the time of the mixing. The transparent solution means that “the components are uniformly dissolved without any impurities and the like.” In each of Comparative Example 1 and Comparative Example 2 in which the monomer represented by the formula (E) was changed to TRIS {the compound different from the monomer represented by the formula (E), the compound serving as a monomer having a silicone moiety}, a transparent solution could not be obtained because the components became insoluble at the time of their mixing. It was assumed that the monomer represented by the formula (E) had a moiety derived from succinic acid, and hence its compatibility with the drug (Q) was improved to enable the preparation of a transparent solution.

### Example 3 and Example 4

Next, a circular film-shaped sample was prepared by using each of Example 1 and Example 2, and transparency and oxygen permeability in the film-shaped sample needed at the time of the production of a drug sustained-releasing medical contact lens were evaluated (Example 3 and Example 4). Reference Example 1 obtained by removing the drug (Q) from Example 3 and Reference Example 2 obtained by removing the drug (Q) from Example 4 were separately prepared as comparison objects at the time of the performance of the oxygen permeability evaluation. The results of the evaluations of Example 3, Example 4, Reference Example 1, and Reference Example 2 are shown in Table 2 together with their compositions.

### Example 3

0.5 Part by mass of ethylene glycol dimethacrylate (EGDMA) and 0.5 part by mass of azobisisobutyronitrile (AIBN) were added to the solution prepared in Example 1, and were mixed and dissolved therein. The solution was poured into a cell sandwiched between a glass plate and a polypropylene plate with a polyethylene terephthalate sheet having a thickness of 0.1 mm as a spacer, and an oven was purged with nitrogen. Next, the solution was heated at 100° C. for 2 hours to be polymerized. After the polymerization, the polyethylene terephthalate sheet was removed from the die, and the resultant polymer was immersed in a solution containing ethanol and ion-exchanged water at a ratio of 3:1 for 12 hours. Further, the polymer was immersed in ion-exchanged water for 12 hours to produce a water-containing film. The produced water-containing film was prepared to a shape needed for each measurement. The transparency of the film was visually observed, and it was confirmed that the film was transparent. Further, the oxygen permeability of the circular film-shaped sample was measured in water at 25° C. (with K-316, manufactured by Tsukuba Rika Seiki). The result of the oxygen permeability measurement is shown in Table 2.

### Example 4

A film was prepared in accordance with the same procedure as that of Example 3 except that Example 2 was used instead of Example 1. It was confirmed that the film of Example 4 was transparent. The result of the oxygen permeability measurement of Example 4 is shown in Table 2.

### Reference Example 1 and Reference Example 2

Films were each prepared in accordance with the same procedure as that of Example 3 except that components whose kinds and amounts were shown in Table 2 were used. The results of the transparency evaluations of the films and the results of the oxygen permeability measurements thereof are shown in Table 2.

It was found that each of the films of Example 3 and Example 4, and Reference Example 1 and Reference Example 2 was transparent and hence suitable for the production of a contact lens. In comparison between the results of the oxygen permeability measurements of Example 3 and Example 4, and those of Reference Example 1 and Reference Example 2, no difference in oxygen permeability was found irrespective of the presence or absence of the drug (Q), and hence each of the films showed satisfactory oxygen permeability. It was confirmed from the foregoing that each of the drug sustained-releasing medical contact lenses of Example 3 and Example 4 was a contact lens excellent in transparency and oxygen permeability.

TABLE 1

| | | | | Comparative | Comparative |
|---|---|---|---|---|---|
| | | Example 1 | Example 2 | Example 1 | Example 2 |
| Polymerization | Monomer | 40 | 60 | | |
| composition | represented by | | | | |
| (part(s) by mass) | formula (E) | | | | |
| | TRIS | | | 40 | 60 |
| | HEMA | 60 | 40 | 60 | 40 |
| | (Total) | 100 | 100 | 100 | 100 |
| | Drug (Q): | 0.005 | 0.005 | 0.005 | 0.005 |
| | latanoprost | | | | |
| Physical | Solution | Transparent | Transparent | Insoluble | Insoluble |
| property | transparency | | | | |

TABLE 2

| | | | | Reference | Reference |
|---|---|---|---|---|---|
| | | Example 3 | Example 4 | Example 1 | Example 2 |
| Film shaped | Monomer represented by | 40 | 60 | 40 | 60 |
| sample | formula (E) | | | | |
| composition | TRIS | | | | |
| (part(s) by mass) | HEMA | 60 | 40 | 60 | 40 |
| | EGDMA | 0.5 | 0.5 | 0.5 | 0.5 |
| | AIBN | 0.5 | 0.5 | 0.5 | 0.5 |
| | (Total) | 101 | 101 | 101 | 101 |
| | Drug (Q): latanoprost | 0.005 | 0.005 | | |
| Film-shaped | Polymer | 99.995 | 99.995 | 100.000 | 100.000 |
| sample | Drug (Q): latanoprost | 0.005 | 0.005 | | |
| composition | (Total) | 100.000 | 100.000 | 100.000 | 100.000 |
| (mass %) | | | | | |
| Physical | Film transparency | Transparent | Transparent | Transparent | Transparent |
| property | Oxygen permeability* | 57 | 60 | 57 | 60 |

In Table 2, “*” represents a unit of ×10⁻¹¹ (cm²/sec)×(mLO₂/(mL×mmHg)).

Monomer represented by the formula (E): 3-[tris(trimethylsiloxy)silyl]propyl methacryloyloxyethylsuccinate

TRIS: 3-tris(trimethylsilyl)propyl methacrylate

HEMA: 2-hydroxyethyl methacrylate

EGDMA: ethylene glycol dimethacrylate

AIBN: azobisisobutyronitrile

### Example 5 and Example 6

Further, the following contact lens drug sustained-releasability test was performed on each of the contact lenses of Reference Example 1 and Reference Example 2, and the results of their evaluations were defined as the results of Example 5 and Example 6, respectively.

<Contact Lens Drug Sustained-Releasability Test>

The contact lens drug sustained-releasability test was performed in accordance with the following procedure.

(1) 8.3 g of sodium chloride, 5.993 g of sodium hydrogen phosphate dodecahydrate, and 0.528 g of sodium dihydrogen phosphate dihydrate were weighed in a measuring flask, and water was added to dissolve the components so that the total volume became 1,000 mL. Thus, ISO physiological saline was obtained.
(2) 80 g of water was weighed, and 0.75 g of sodium chloride, 0.263 g of disodium hydrogen phosphate, 0.141 g of sodium dihydrogen phosphate, 0.01 g of benzalkonium chloride, 0.1 g of polysorbate 80, and 0.005 g of latanoprost were sequentially weighed and added thereto. Water was further added to the liquid so that the total amount became 100 g. Thus, a latanoprost solution was obtained.
(3) 10 mL of the liquid obtained in the (2) and the film of Reference Example 1 were loaded into a glass petri dish, and the film was immersed in the liquid overnight.
(4) After that, 10 mL of the ISO physiological saline was loaded into another glass petri dish.
(5) The film of Reference Example 1 after the immersion in the (3) was removed, and was sufficiently rinsed with the ISO physiological saline (the film corresponds to the drug sustained-releasing medical contact lens of the present invention having retained therein the drug (Q)).
(6) The film of Reference Example 1 sufficiently rinsed in the (5) was loaded into the glass petri dish in the (4).
(7) Immediately after the (6), the liquid portion was sampled in amounts of 0.5 mL each.
(8) Further, the sampling was performed 1 hour, 2 hours, 4 hours, 8 hours, 24 hours, and 32 hours after the (7).
(9) The sampled liquids were subjected to quantitative analysis by using <Analysis Conditions> described below.
(10) After the analysis, the drug sustained-releasability of Reference Example 1 was evaluated by using <Drug Sustained-release Ratio Calculation Equation> described below, and the results of the evaluation were defined as the results of Example 5.

The contact lens drug sustained-releasability test was also performed on Reference Example 2 by the above-mentioned procedure, and the results of the evaluation were defined as the results of Example 6.

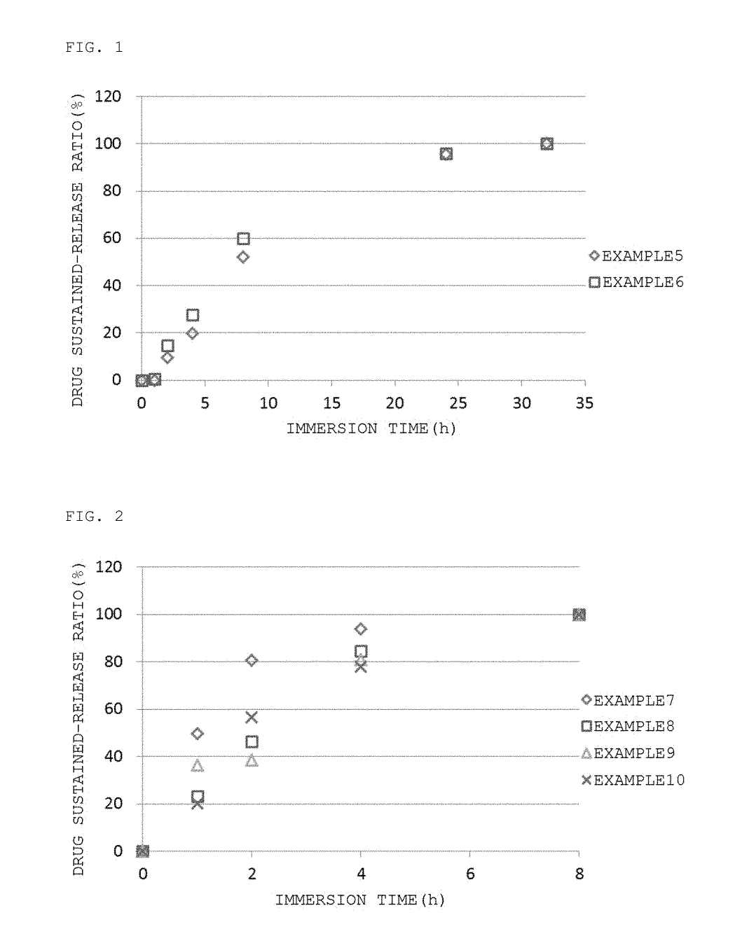

The results of Example 5 and Example 6 are shown in FIG. 1.

<Analysis Conditions>

Column: A stainless-steel tube having an inner diameter of 4.6 mm and a length of 150 mm is filled with 5-micrometer octadecylsilylated silica gel for liquid chromatography.

Column oven: A constant temperature around 25° C.

Injection amount: 20 μL

Flow rate: The flow rate is adjusted so that the retention time of latanoprost may be about 8 minutes.

Detector: An ultraviolet-visible spectral detector (210 nm)

Mobile phase: A potassium dihydrogen phosphate solution was obtained by weighing 3.40 g of potassium dihydrogen phosphate and dissolving the potassium dihydrogen phosphate in water so that the total volume precisely became 500 mL. 300 mL of the solution was weighed and 700 mL of acetonitrile was added thereto, followed by the adjustment of the pH of the mixture to 3.0 with 10% phosphoric acid. Thus, the mobile phase was obtained.

<Drug Sustained-Release Ratio Calculation Equation>

Drug sustained-release ratio (%)=(peak area of latanoprost at each sampling time)/(peak area of latanoprost at sampling 32 hours after the (7))×100

As a result of the drug sustained-releasability test, each of Example 5 and Example 6 had a linear drug sustained-releasability in substantial proportion to an immersion time in the ISO physiological saline (FIG. 1). It was confirmed from the foregoing that each of Example 5 and Example 6 had drug sustained-releasability for latanoprost up to 32 hours after the (7).

It was found from the foregoing that the contact lens of the present invention was excellent in oxygen permeability and film transparency, and was excellent as a drug sustained-releasing medical contact lens.

### Example 7 to Example 10

Contact lenses (film-shaped samples) each using a drug sustained-releasing medical contact lens solution of the present invention were prepared in accordance with the same procedures as those of Example 1 and Example 3 except that components whose kinds and amounts were shown in Table 3 were used. The transparency of each of the prepared films of Example 7 to Example 10 was observed, and the results were as shown in Table 3. In addition, the oxygen permeability of each of the circular film-shaped samples was measured in water at 25° C. in the same manner as in Example 3. The results of the measurement are shown in Table 3.

TABLE 3

| | | Example 7 | Example 8 | Example 9 | Example 10 |
|---|---|---|---|---|---|
| Film-shaped | Monomer represented by | 59.6 | 59.6 | 59.6 | 59.6 |
| sample | formula (F1) | | | | |
| composition | MFC | | 5 | 10 | 15 |
| (part(s) by mass) | HEA | 24.8 | 19.8 | 14.8 | 9.8 |
| | NVP | 14.9 | 14.9 | 14.9 | 14.9 |
| | EGDMA | 0.5 | 0.5 | 0.5 | 0.5 |
| | AIBN | 0.2 | 0.2 | 0.2 | 0.2 |
| | (Total) | 100 | 100 | 100 | 100 |
| Physical | Film transparency | Transparent | Transparent | Transparent | Transparent |
| property | Oxygen permeability* | 76 | 77 | 78 | 80 |

In Table 3, “*” represents a unit of ×10⁻¹¹ (cm²/sec)×(mLO₂/(mL×mmHg)).

Monomer represented by the formula (F1): [tris(trimethylsiloxy)silyl]propyl=3-(2-hydroxyethoxy)carbonyl-3-butenate

MPC: 2-methacryloyloxyethyl phosphorylcholine

HEA: 2-hydroxyethyl acrylate

NVP: N-vinylpyrrolidone

EGDMA: ethylene glycol dimethacrylate

AIBN: azobisisobutyronitrile

As a result of the film transparency observation and the oxygen permeability measurement, each of the films of Example 7 to Example 10 was transparent and hence suitable for the production of a contact lens. In addition, Example 10 was found to have the most excellent oxygen permeability.

Further, a contact lens drug sustained-releasability test up to 8 hours after the (7) was performed on each of the polymers of Example 7 to Example 10 each serving as a contact lens excellent in oxygen permeability with reference to <Contact Lens Drug Sustained-releasability Test>.

<Drug Sustained-release Ratio Calculation Equation 2> described below was used in the calculation of the drug sustained-release ratio of each of Example 7 to Example 10.

### <Drug Sustained-Release Ratio Calculation Equation 2>

Drug sustained-release ratio (%)=(peak area of latanoprost at each sampling time)/(peak area of latanoprost at sampling 8 hours after the (7))×100

The results of Example 7 to Example 10 are shown in FIG. 2. As is apparent from FIG. 2, it was confirmed that the polymers of Example 7 to Example 10 each had drug sustained-releasability up to 8 hours after the (7). It was confirmed from the foregoing that the polymers of Example 7 to Example 10 each also had drug sustained-releasability for latanoprost.

It was found from the foregoing that the contact lens of the present invention was excellent in drug sustained-releasability, and was hence useful as a drug sustained-releasing medical contact lens.

### INDUSTRIAL APPLICABILITY

There can be provided a drug sustained-releasing medical contact lens in which a component that is water-insoluble and has pharmacological activity is retained to impart drug sustained-releasability, and which is excellent in oxygen permeability.

1-6. (canceled)

7. A drug sustained-releasing method, including using a drug sustained-releasing medical contact lens in which the content of the polymer (P) having the constituent unit represented by the formula (1) or the formula (F1) is from 90 mass % to 99.99999 mass %, and the content of the drug (Q) having a solubility in water of from 0.00001% to 3.3% is from 0.00001 mass % to 10 mass % in a mammal including a human: 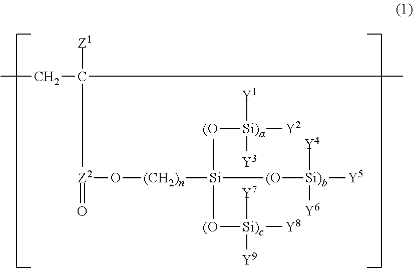
where Y¹ to Y⁹ each independently represent an alkyl group having 1 to 4 carbon atoms, n represents an integer of from 0 to 3, a, b, and c each independently represent an integer of 0 or 1, Z¹ represents a hydrogen atom, a methyl group, or CH₂—C(═O)—X, X represents a monovalent organic group having 2 to 6 carbon atoms that contain one or more hydroxyl groups, and that contain one oxygen atom, and that may contain nitrogen atom in a main chain thereof, and Z² represents a carbon atom or C(═O)—O—(CH₂)₂—O—C(═O)—(CH₂)₂—C.

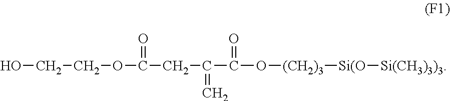

8. A drug sustained-releasing method according to claim 7, wherein the constituent unit represented by the formula (1) is represented by the formula (A) or the formula (B): 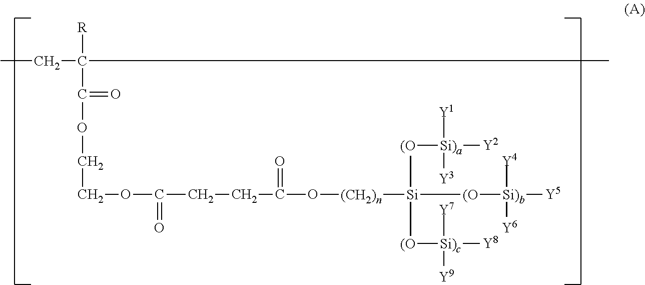
where Y¹ to Y⁹ each independently represent an alkyl group having 1 to 4 carbon atoms, n represents an integer of from 0 to 3, a, b, and c each independently represent an integer of 0 or 1, and R represents a hydrogen atom or a methyl group; 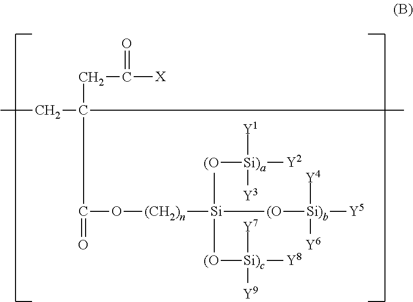
where Y¹ to Y⁹ each independently represent an alkyl group having 1 to 4 carbon atoms, n represents an integer of from 0 to 3, a, b, and c each independently represent an integer of 0 or 1, and X represents a monovalent organic group having 2 to 6 carbon atoms that contain one or more hydroxyl groups, and that contain one oxygen atom, and that may contain nitrogen atom in a main chain thereof.

9. A drug sustained-releasing method according to claim 7, wherein X represents any one of the following groups (a) to (f).

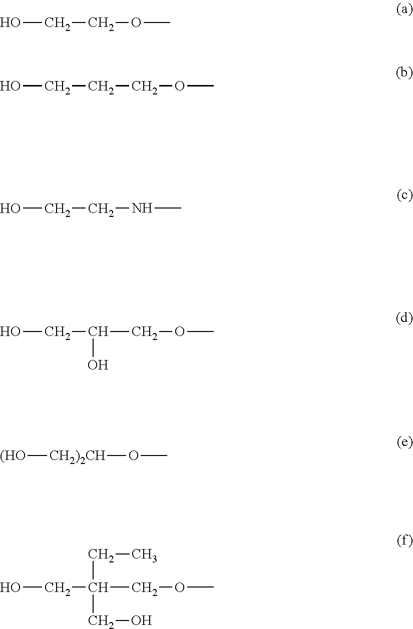

10. A drug sustained-releasing method according to claim 8, wherein X represents any one of the following groups (a) to (f).

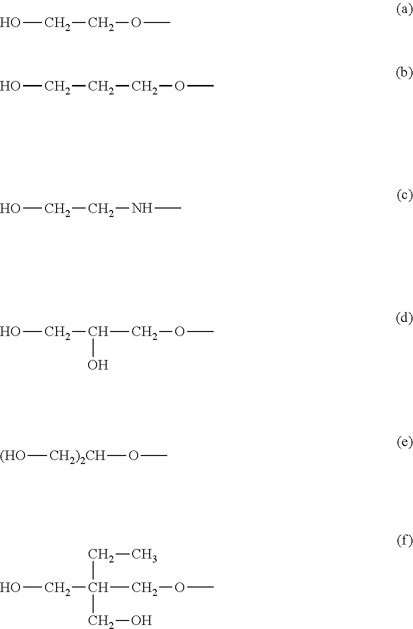

11. A drug sustained-releasing method according to claim 7, wherein the drug (Q) comprises one or more kinds of drug (Q) selected from the group consisting of a drug for glaucoma treatment, an antiallergic drug, an antiinflammatory drug, an adrenocortical steroid drug, an antibacterial drug, an anti-cataract drug, a drug for corneal disorder treatment, an antiviral drug, a mydriatic, a cycloplegic, and a vitamin agent.

12. A drug sustained-releasing method according to claim 8, wherein the drug (Q) comprises one or more kinds of drug (Q) selected from the group consisting of a drug for glaucoma treatment, an antiallergic drug, an antiinflammatory drug, an adrenocortical steroid drug, an antibacterial drug, an anti-cataract drug, a drug for corneal disorder treatment, an antiviral drug, a mydriatic, a cycloplegic, and a vitamin agent.

13. A drug sustained-releasing method according to claim 9, wherein the drug (Q) comprises one or more kinds of drug (Q) selected from the group consisting of a drug for glaucoma treatment, an antiallergic drug, an antiinflammatory drug, an adrenocortical steroid drug, an antibacterial drug, an anti-cataract drug, a drug for corneal disorder treatment, an antiviral drug, a mydriatic, a cycloplegic, and a vitamin agent.

14. A drug sustained-releasing method according to claim 10, wherein the drug (Q) comprises one or more kinds of drug (Q) selected from the group consisting of a drug for glaucoma treatment, an antiallergic drug, an antiinflammatory drug, an adrenocortical steroid drug, an antibacterial drug, an anti-cataract drug, a drug for corneal disorder treatment, an antiviral drug, a mydriatic, a cycloplegic, and a vitamin agent.

15. A drug sustained-releasing method according to claim 7, wherein the polymer (P) comprises a polymer having a constituent unit based on 3-[tris(trimethylsiloxy)silyl]propyl methacryloyloxyethylsuccinate, and the drug (Q) comprises latanoprost.

16. A drug sustained-releasing method according to claim 7, wherein the polymer (P) comprises a polymer having a constituent unit represented by the formula (F1), and the drug (Q) comprises latanoprost.

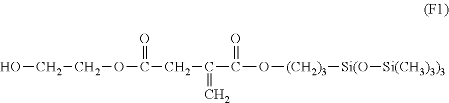

17. A drug sustained-releasing medical contact lens, comprising:
a polymer (P) having a constituent unit represented by the formula (1) or the formula (F1); and
a drug (Q) having a solubility in water of from 0.00001% to 3.3%,
wherein a content of the polymer (P) is from 90 mass % to 99.99999 mass %, and a content of the drug (Q) is from 0.00001 mass % to 10 mass %: 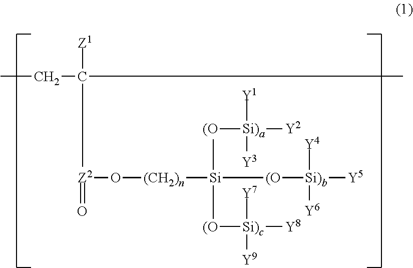
where Y¹ to Y⁹ each independently represent an alkyl group having 1 to 4 carbon atoms, n represents an integer of from 0 to 3, a, b, and c each independently represent an integer of 0 or 1, Z¹ represents a hydrogen atom, a methyl group, or CH₂—C(═O)—X, X represents a monovalent organic group having 2 to 6 carbon atoms that contain one or more hydroxyl groups, and that contain one oxygen atom, and that may contain nitrogen atom in a main chain thereof, and Z² represents a carbon atom or C(═O)—O—(CH₂)₂—O—C(═O)—(CH₂)₂—C.

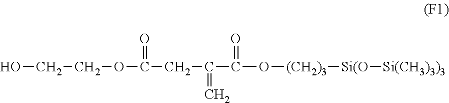

18. A drug sustained-releasing medical contact lens according to claim 17, wherein the constituent unit represented by the formula (1) is represented by the formula (A) or the formula (B): 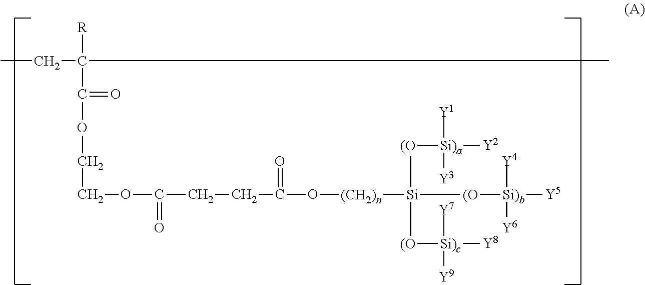
where Y¹ to Y⁹ each independently represent an alkyl group having 1 to 4 carbon atoms, n represents an integer of from 0 to 3, a, b, and c each independently represent an integer of 0 or 1, and R represents a hydrogen atom or a methyl group; 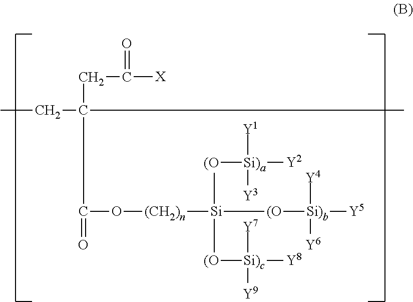
where Y¹ to Y⁹ each independently represent an alkyl group having 1 to 4 carbon atoms, n represents an integer of from 0 to 3, a, b, and c each independently represent an integer of 0 or 1, and X represents a monovalent organic group having 2 to 6 carbon atoms that contain one or more hydroxyl groups, and that contain one oxygen atom, and that may contain nitrogen atom in a main chain thereof.

19. A drug sustained-releasing medical contact lens according to claim 17, wherein X represents any one of the following groups (a) to (f).

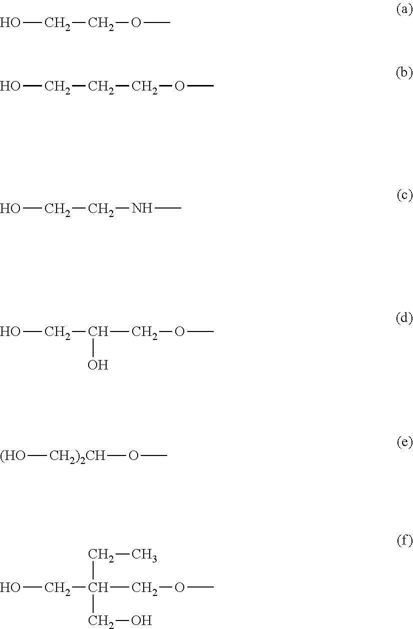

20. A drug sustained-releasing medical contact lens according to claim 17, wherein the drug (Q) comprises one or more kinds of drug (Q) selected from the group consisting of a drug for glaucoma treatment, an antiallergic drug, an antiinflammatory drug, an adrenocortical steroid drug, an antibacterial drug, an anti-cataract drug, a drug for corneal disorder treatment, an antiviral drug, a mydriatic, a cycloplegic, and a vitamin agent.

21. A drug sustained-releasing medical contact lens according to claim 17, wherein the polymer (P) comprises a polymer having a constituent unit based on 3-[tris(trimethylsiloxy)silyl]propyl methacryloyloxyethylsuccinate, and the drug (Q) comprises latanoprost.

22. A drug sustained-releasing medical contact lens according to claim 17, wherein the polymer (P) comprises a polymer having a constituent unit represented by the formula (F1), and the drug (Q) comprises latanoprost. [chemical structure]
